(19) United States
(12) Patent Application Publication
(10) Pub. No.: US 2025/0288816 A1
(43) Pub. Date: Sep. 18, 2025

(54) TRIALING SYSTEM FOR SACRAL NEUROMODULATION THERAPY

(71) Applicants: Alexander Jueshyan Yeh, Los Altos Hills, CA (US); Vivian Thalia Aralis, Redwood City, CA (US); Avner Kornfeld, San Jose, CA (US); Peter Velasco Obico, Santa Clara, CA (US); Mike Clinton Holzbaur, Mountain View, CA (US)

(72) Inventors: Alexander Jueshyan Yeh, Los Altos Hills, CA (US); Vivian Thalia Aralis, Redwood City, CA (US); Avner Kornfeld, San Jose, CA (US); Peter Velasco Obico, Santa Clara, CA (US); Mike Clinton Holzbaur, Mountain View, CA (US)

(21) Appl. No.: 19/079,210

(22) Filed: Mar. 13, 2025

Related U.S. Application Data
(60) Provisional application No. 63/565,441, filed on Mar. 14, 2024.

(51) Int. Cl. A61N 1/372; A61N 1/05; A61N 1/36; A61N 1/378

(52) U.S. Cl. CPC A61N 1/37241; A61N 1/05; A61N 1/3605; A61N 1/37217; A61N 1/37247; A61N 1/37252; A61N 1/3787

(57) ABSTRACT

A neurostimulation trial system includes an external neurostimulation therapy controller including a processor circuit, a pulse generator circuit, a ground pad configured to be coupled with an external surface of patient skin tissue, and a transceiver circuit configured to communicate with a programmer device. The system can include at least one lead with an electrode that is configured for implantation at a neural tissue site inside a patient body. In an example, the programmer device is configured to communicate therapy instructions to the external neurostimulation therapy controller and is configured to communicate power and/or therapy instructions to an implanted neurostimulation device.

### CROSS-REFERENCE TO RELATED APPLICATIONS

This application is related to and claims priority to U.S. Provisional Application No. 63/565,441, filed on Mar. 14, 2024, and entitled “Trialing System for Sacral Neuromodulation Therapy,” the entirety of which is incorporated herein by reference.

### BACKGROUND

Neurostimulation can be used to treat various disorders including chronic pain, overactive bladder, and other disorders. Implantable neurostimulators, which deliver electrical impulses to specific neural targets, have shown promise in modulating neural activity to alleviate symptoms.

Patient responses to neurostimulation therapy varies. Some pre-implantation evaluation methods focus on optimizing patient comfort and minimizing procedural risks while striving to enhance the predictability of long-term therapeutic efficacy. The evolution of these evaluation methods continues to play a crucial role in personalized medical treatments, aiming to ensure that the benefits of neurostimulation are fully realized by patients.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates generally an example of a neuromodulation trial system.

FIG. 2A illustrates generally a first perspective view of an example of an external stimulation controller.

FIG. 2B illustrates generally a second perspective view of an example of an external stimulation controller.

FIG. 3A through FIG. 3G illustrate generally various examples of a housing of an external stimulation controller.

FIG. 4 illustrates generally an example of a portion of a lead assembly.

FIG. 5A illustrates generally a perspective view of an example of a foramen needle assembly.

FIG. 5B illustrates generally an exploded view of a foramen needle assembly.

FIG. 6 illustrates generally an example of a neuromodulation system with a permanent implantable device.

FIG. 7 illustrates generally an example of a portion of an external system.

FIG. 8 illustrates generally an example of a portion of a wireless transmitter.

FIG. 9 illustrates generally an example a portion of a wireless transmitter.

FIG. 10 illustrates generally an example of a portion of a wireless receiver.

### DETAILED DESCRIPTION

In the following description that includes examples of different trial systems and system components for neurostimulation, reference is made to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. The present inventors contemplate examples using any combination or permutation of those elements shown or described (or one or more aspects thereof), either with respect to a particular example (or one or more aspects thereof), or with respect to other examples (or one or more aspects thereof) shown or described herein.

A neurostimulator trial system for use during a trial phase is designed to provide a temporary period of neural stimulation to help determine the therapeutic benefits for a patient prior to committing to a permanent implant. The trial phase can help determine the patient's responsiveness to neurostimulation, thereby avoiding unnecessary risks and costs associated with a permanent implant in therapy non-responders.

A trial system generally comprises internal (i.e., implanted, or partially implanted) components and external components. Internally, one or more temporary leads, such as having one or more electrodes each, can be positioned within a target neural region. Externally, a power source (e.g., a battery) is coupled with an external stimulation controller (sometimes referred to herein as an external stimulator) that can be worn or used by the patient.

The electrode lead or leads can be placed using a minimally invasive approach, sometimes under local anesthesia and with the assistance of imaging such as fluoroscopy. After placement, the lead or leads are connected to the controller. The controller is then programmed to deliver precise electrical impulses to the targeted neural structures.

The duration of a trial is typically about one week. This timeframe allows for a comprehensive evaluation of the stimulation effects on the patient's symptoms.

The patient's response to the trial stimulation can be monitored. This monitoring can include maintaining a record of pain levels, alterations in medication, and any changes in the patient's functional abilities or overall quality of life.

When the trial period concludes, the temporary leads are removed. If the trial indicates a positive response, then the patient may proceed to receive a permanent implanted neurostimulator. In an example, the permanent device comprises a midfield implantable device as described herein; other devices can similarly be used. Conversely, if the trial does not yield a favorable outcome, then the leads are removed, and alternative therapeutic options can be explored.

In an example, systems and methods discussed herein provide a trial neurostimulation system that includes an external stimulator designed for ambulatory stimulation over a multiple-day trial period. The external stimulator comprises a battery and circuitry to receive therapy instructions (e.g., from an external wireless transmitter) and generate or provide neurostimulation signals to one or more electrodes attached to the stimulator and the patient. In an example, the external stimulator includes or uses instances of some or all of the same circuitry (e.g., an Application-Specific Integrated Circuit (ASIC)) or the same or similar circuit functionality as is used in a corresponding system that includes a permanent implant, thereby ensuring consistency within the treatment ecosystem.

The trial system allows programming or control using one or more of a wireless transmitter, patient device, or clinician programmer. In some examples, the same wireless transmitter, patient device, and/or clinician programmer can be used for intraoperative and post-operative programming adjustments.

In an example, the external stimulator is configured to provide substantially continuous or intermittent stimulation and is powered by a battery with a service life of multiple days (e.g., at least seven days). In an example, the external stimulator adheres directly to the skin using a conductive pad (e.g., a ground pad), eliminating the need for a separate belt, and is designed to be a fully hardware-based solution without the requirement for additional firmware or software.

In an example, the external stimulator repurposes parts of the permanent implant device system as test infrastructure. The external stimulator can interface with various components during intraoperative testing and throughout the trial period, including the ground pad, trialing leads for unilateral or bilateral testing, and the wireless transmitter, patient device, or clinician programmer. The system can optionally include procedural accessories such as foramen needles, clip lead cables, and a range of surgical supplies to support the trial system implantation and configuration processes.

In an example, the external stimulator includes an enclosure. The enclosure optionally includes or is coupled to a tissue interface layer or ground pad, light pipe, cable, and an ECG sensor or terminal. The enclosure can have a minimum ingress protection rating of, e.g., IP22. The system features and design aim to streamline the trial process for neurostimulation therapy, providing a user-friendly and efficient solution for evaluating the potential benefits of a permanent implant.

In an example, a permanent implantable device can be provided following a successful trial with the trial system. The permanent implantable device can comprise one or more devices configured to wirelessly receive power and/or data, such as via midfield signals. Midfield technology can provide power to a deeply implanted electrostimulation device from an external power source located on or near a tissue surface, such as at an external surface of a user's skin. The user can be a clinical patient or other user. The midfield powering technology can have one or more advantages over conventional implantable pulse generators. For example, a conventional pulse generator can have one or more relatively large, implanted batteries. Midfield devices, in contrast, can include relatively small power storage elements or battery cells that can be configured to receive and store relatively small amounts of power. A midfield device can include one or more electrodes integrated in a unitary implantable package. Thus, in some examples, a midfield-powered device can provide a simpler implant procedure over other devices, which can lead to a lower cost and a lower risk of infection or other implant complications. Another advantage of midfield powering includes the efficiency with which power can be transferred to the implanted device. The ability to focus energy from a midfield transmitter to a midfield receiver device can allow for an increase in an amount of power transferred to, and ultimately received by, the implanted device.

An advantage of using midfield powering technology can include a main battery or power source being provided externally to the patient, and thus low power consumption and high efficiency circuitry requirements of conventional battery-powered implantable devices can be relaxed. Another advantage of using midfield powering technology can include an implanted device that can be physically smaller than a battery-powered device. Midfield powering technology can thus help enable better patient tolerance and comfort along with potentially lower costs to manufacture and/or to implant in patient tissue.

In one or more examples that include using a midfield wireless transmitter or receiver, tissue can act as a dielectric to tunnel energy. Coherent interference of propagating modes can confine a field at a focal plane to less than a corresponding vacuum wavelength, for example, with a spot size subject to a diffraction limit in a high-index material. In one or more examples, a receiver (e.g., implanted in tissue) positioned at such a high energy density region, can be one or more orders of magnitude smaller than a conventional nearfield implantable receiver, or can be implanted more deeply in tissue (e.g., greater than 1 cm in depth). In one or more examples, a transmitter source described herein can be configured to provide electromagnetic energy to various target locations, including for example to one or more deeply implanted devices. In an example, the energy can be provided to a location with greater than about a few millimeters of positioning accuracy. That is, a transmitted power or energy signal can be directed or focused to a target location that is within about one wavelength of the signal in tissue. Such energy focusing is substantially more accurate than the focusing available via traditional inductive means and is sufficient to provide adequate power to a receiver. In other wireless powering approaches such as using nearfield coupling (inductive coupling and its resonant enhanced derivatives), evanescent components outside tissue (e.g., near the source) remain evanescent inside tissue, which does not allow for effective depth penetration. Unlike nearfield coupling, energy from a midfield source is primarily carried in propagating modes and, as a result, an energy transport depth is limited by environmental losses rather than by intrinsic decay of the nearfield. Energy transfer implemented with these characteristics can be at least two to three orders of magnitude more efficient than nearfield systems.

One or more of the systems, apparatuses, and methods discussed herein can be used to help treat a patient disorder. Disorders such as fecal or urinary incontinence (e.g., overactive bladder) can be treated for example by stimulating the tibial nerve or any branch of the tibial nerve, such as but not limited to the posterior tibial nerve, one or more nerves or nerve branches originating from the sacral plexus, including but not limited to S1-S4, the tibial nerve, and/or the pudendal nerve. Urinary incontinence may be treated by stimulating one or more of muscles of the pelvic floor, nerves innervating the muscles of the pelvic floor, internal urethral sphincter, external urethral sphincter, and the pudendal nerve or branches of the pudendal nerve.

One or more of the systems, apparatuses, and methods discussed herein can be used to help treat sleep apnea and/or snoring by stimulating one or more of a nerve or nerve branches of the hypoglossal nerve, the base of the tongue (muscle), phrenic nerve(s), intercostal nerve(s), accessory nerve(s), and cervical nerves C3-C6. Treating sleep apnea and/or snoring can include providing energy to an implant to sense a decrease, impairment, or cessation of breathing (such as by measuring oxygen saturation).

One or more of the systems, apparatuses, and methods discussed herein can be used to help treat vaginal dryness, such as by stimulating one or more of Bartholin gland(s), Skene's gland(s), and inner wall of vagina. One or more of the systems, apparatuses, and methods discussed herein can be used to help treat migraines or other headaches, such as by stimulating one or more of the occipital nerve, supraorbital nerve, C2 cervical nerve, or branches thereof, and the frontal nerve, or branches thereof. One or more of the systems, apparatuses, and methods discussed herein can be used to help treat post-traumatic stress disorder, hot flashes, and/or complex regional pain syndrome such as by stimulating one or more of the stellate ganglion and the C4-C7 of the sympathetic chain.

One or more of the systems, apparatuses, and methods discussed herein can be used to help treat neuralgia (e.g., trigeminal neuralgia), such as by stimulating one or more of the sphenopalatine ganglion nerve block, the trigeminal nerve, or branches of the trigeminal nerve. One or more of the systems, apparatuses, and methods discussed herein can be used to help treat dry mouth (e.g., caused by side effects from medications, chemotherapy or radiation therapy cancer treatments, Sjogren's disease, or by other cause of dry mouth), such as by stimulating one or more of Parotid glands, submandibular glands, sublingual glands, submucosa of the oral mucosa in the oral cavity within the tissue of the buccal, labial, and/or lingual mucosa, the soft palate, the lateral parts of the hard palate, and/or the floor of the mouth and/or between muscle fibers of the tongue, Von Ebner glands, glossopharyngeal nerve (CN IX), including branches of CN IX, including otic ganglion, a facial nerve (CN VII), including branches of CN VII, such as the submandibular ganglion, and branches of T1-T3, such as the superior cervical ganglion.

One or more of the systems, apparatuses, and methods discussed herein can be used to help treat a transected nerve, such as by sensing electrical output from the proximal portion of a transected nerve and delivering electrical input into the distal portion of a transected nerve, and/or sensing electrical output from the distal portion of a transected nerve and delivering electrical input into the proximal portion of a transected nerve. One or more of the systems, apparatuses, and methods discussed herein can be used to help treat cerebral palsy, such as by stimulating one or more muscles or one or more nerves innervation one or more muscles affected in a patient with cerebral palsy. One or more of the systems, apparatuses, and methods discussed herein can be used to help treat erectile dysfunction, such as by stimulating one or more of pelvic splanchnic nerves (S2-S4) or any branches thereof, the pudendal nerve, cavernous nerve(s), and inferior hypogastric plexus.

One or more of the systems, apparatuses, and methods discussed herein can be used to help treat menstrual pain, such as by stimulating one or more of the uterus and the vagina. One or more of the systems, apparatuses, and methods discussed herein can be used as an intrauterine device, such as by sensing one or more PH and blood flow or delivering current or drugs to aid in contraception, fertility, bleeding, or pain. One or more of the systems, apparatuses, and methods discussed herein can be used to incite human arousal, such as by stimulating female genitalia, including external and internal, including clitoris or other sensory active parts of the female, or by stimulating male genitalia.

One or more of the systems, apparatuses, and methods discussed herein can be used to help treat hypertension, such as by stimulating one or more of a carotid sinus, left or right cervical vagus nerve, or a branch of the vagus nerve. One or more of the systems, apparatuses, and methods discussed herein can be used to help treat paroxysmal supraventricular tachycardia, such as by stimulating one or more of trigeminal nerve or branches thereof, anterior ethmoidal nerve, and the vagus nerve. One or more of the systems, apparatuses, and methods discussed herein can be used to help treat vocal cord dysfunction, such as by sensing the activity of a vocal cord and the opposite vocal cord or just stimulating one or more of the vocal cords by stimulating nerves innervating the vocal cord, the left and/or Right recurrent laryngeal nerve, and the vagus nerve.

One or more of the systems, apparatuses, and methods discussed herein can be used to help repair tissue, such as by stimulating tissue to do one or more of enhancing microcirculation and protein synthesis to heal wounds and restoring integrity of connective and/or dermal tissues. One or more of the systems, apparatuses, and methods discussed herein can be used to help asthma or chronic obstructive pulmonary disease, such as by one or more of stimulating the vagus nerve or a branch thereof, blocking the release of norepinephrine and/or acetylcholine and/or interfering with receptors for norepinephrine and/or acetylcholine.

One or more of the systems, apparatuses, and methods discussed herein can be used to help treat cancer, such as by stimulating, to modulate one or more nerves near or in a tumor, such as to decrease the sympathetic innervation, such as epinephrine/NE release, and/or parasympathetic innervation. One or more of the systems, apparatuses, and methods discussed herein can be used to help treat diabetes, such as by powering a sensor inside the human body that detects parameters of diabetes, such as a glucose level or ketone level and using such sensor data to adjust delivery of exogenous insulin from an insulin pump. One or more of the systems, apparatuses, and methods discussed herein can be used to help treat diabetes, such as by powering a sensor inside the human body that detects parameters of diabetes, such as a glucose level or ketone level, and using a midfield coupler to stimulate the release of insulin from islet beta cells.

One or more of the systems, apparatuses, and methods discussed herein can be used to help treat neurological conditions, disorders or diseases (such as Parkinson's disease (e.g., by stimulating an internus or nucleus of the brain), Alzheimer's disease, Huntington's disease, dementia, Creutzfeldt-Jakob disease, epilepsy (e.g., by stimulating a left cervical vagus nerve or a trigeminal nerve), post-traumatic stress disorder (PTSD) (e.g., by stimulating a left cervical vagus nerve), or essential tremor, such as by stimulating a thalamus), neuralgia, depression, dystonia (e.g., by stimulating an internus or nucleus of the brain), phantom limb (e.g., by stimulating an amputated nerve, such an ending of an amputated nerve), dry eyes (e.g., by stimulating a lacrimal gland), arrhythmia (e.g., by stimulating the heart), a gastrointestinal disorder, such as obesity, gastroesophageal reflux, and/or gastroparesis, such as by stimulating a C1-C2 occipital nerve or deep brain stimulation (DBS) of the hypothalamus, an esophagus, a muscle near sphincter leading to the stomach, and/or a lower stomach, and/or stroke (e.g., by subdural stimulation of a motor cortex). Using one or more examples discussed herein, stimulation can be provided continuously, on demand (e.g., as demanded by a physician, patient, or other user), or periodically.

In an example, a neuromodulation trial system can be provided or used by a patient before the patient receives a permanent implantable device. A trial system electrode or the electrode(s) of a permanent implantable device can be situated several centimeters (e.g., one to five centimeters, or more) below a tissue interface, that is, below a surface of the skin. In one or more examples, electrode(s) of a trial lead or other implantable device can be situated between about 2 centimeters and 4 centimeters, about 3 centimeters, between about 1 centimeter and five centimeters, less than 1 centimeter, about two centimeters, or other distance below the surface of the skin. The depth of implantation can depend on the intended use. For example, to treat depression, hypertension, epilepsy, and/or PTSD, the electrode(s) can be situated between about 2 centimeters and about four centimeters below the surface of the skin. In another example, to treat sleep apnea, arrhythmia (e.g., bradycardia), obesity, gastroesophageal reflux, and/or gastroparesis, the electrode(s) can be situated at greater than about 3 centimeters below the surface of the skin. In yet another example, to treat Parkinson's, essential tremors, and/or dystonia the electrode(s) can be situated between about 1 centimeter and about 5 centimeters below the surface of the skin. Yet other examples include situating the electrode(s) between about 1 centimeter and about 2 centimeters below the surface of the skin, such as to treat fibromyalgia, stroke, and/or migraine, at about 2 centimeters to treat asthma, and at about one centimeter or less to treat dry eyes.

Although many embodiments included herein describe devices or methods for providing stimulation (e.g., electrostimulation), the embodiments may be adapted to provide other forms of modulation (e.g., denervation) in addition to or instead of stimulation. In addition, although many embodiments included herein refer to the use of electrodes to deliver therapy, other energy delivery members (e.g., ultrasound transducers or other ultrasound energy delivery members, optical elements) or other therapeutic members or substances (e.g., fluid delivery devices or members to deliver chemicals, drugs, cryogenic fluid, hot fluid or steam, or other fluids) may be used or delivered in other embodiments.

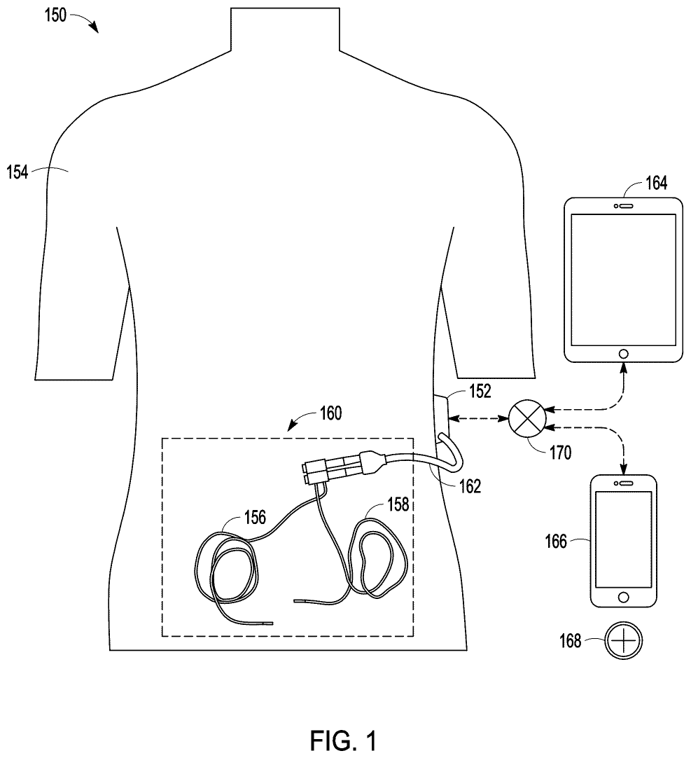

FIG. 1 illustrates generally an example of a neuromodulation trial system 150 applied to, or in use by, a patient. The neuromodulation trial system 150 comprises an external stimulation controller 152 that can be worn by, or otherwise coupled to, the patient. The external stimulation controller 152 can be coupled to one or more neurostimulation leads, such as a first trial lead 156 or a second trial lead 158, using a lead interface 162. In an example, the first trial lead 156 and second trial lead 158 can comprise one or more respective electrodes, or electrode arrays, that are configured for implantation at one or more neural targets in the patient. In a particular example, the leads are configured for bilateral stimulation in a sacral region, at a lower patient posterior 154. Other targets, such as described elsewhere herein, can similarly be used. After implantation of the lead or leads, a protective dressing 160 can be applied over one or more of the first trial lead 156, the second trial lead 158, the lead interface 162, and the external stimulation controller 152. This protective dressing 160 helps maintain sterility and protects the implantation area from external factors that could compromise the integrity of the system during a trial.

In an example, the external stimulation controller 152 comprises a battery, a processor circuit, and a signal generator circuit configured to generate electrical neurostimulation signals that can be provided to the patient using the respective electrodes of the first trial lead 156 or the second trial lead 158. In an example, the external stimulation controller 152 comprises a transceiver circuit configured to communicate data with a wireless interface device 170, with an external programmer or with other device. In an example, the external stimulation controller 152 comprises or is coupled to one or more sensors configured to measure physiologic status information from the patient, such as in response to neurostimulation therapy provided using a signal generator circuit of the external stimulation controller 152.

In an example, the external stimulation controller 152 is configured for data communication with the wireless interface device 170. The wireless interface device 170 can include a wireless receiver and wireless transmitter (e.g., a wireless transmitter-receiver device) that is configured to provide instructions or commands to the external stimulation controller 152 that cause the external stimulation controller 152 to initiate or stop providing a therapy to the patient, or cause the external stimulation controller 152 to update or adjust a therapy parameter. In an example, the wireless interface device 170 is communicatively coupled to a clinician device 164 and/or a patient device 166. The clinician device 164 and/or the patient device 166 can be configured as an external programmer to set or modify parameters of the neurostimulation signals that are generated by the signal generator circuit in the external stimulation controller 152. In an example, the clinician device 164 and/or patient device 166 can provide neurostimulation parameters or instructions to the wireless interface device 170, and the wireless interface device 170 can in turn communicate with the external stimulation controller 152 to cause the external stimulation controller 152 to provide a therapy that corresponds with the parameters. Additionally or alternatively, the clinician device 164 and/or the patient device 166 can provide parameters or instructions directly to the external stimulation controller 152 without using the wireless interface device 170.

The wireless interface device 170 and the external stimulation controller 152 can be configured to communicate wirelessly with each other or with one or more other devices using various communication bands or protocols. In an example, the wireless interface device 170 or the external stimulation controller 152 can use a radio frequency (RF) communication band, such as a Bluetooth protocol, or can use various other wireless communication bands or protocols. The wireless interface device 170 can be configured to communicate with the clinician device 164 and/or the patient device 166 using the same or other wireless communication bands or protocols. For example, the wireless interface device 170 can be configured to use a first protocol to communicate with the external stimulation controller 152 (e.g., via Bluetooth) and use a different second protocol (e.g., via WiFi) to communicate with the clinician device 164 and/or the patient device 166.

In an example, the wireless interface device 170 is the same device that is or can be used to communicate power and data to an implanted neuromodulation device or system. However, when used in the context of the neuromodulation trial system 150, the wireless interface device 170 can be configured to use non-midfield-based transmissions to communicate with the external stimulation controller 152. When used with an implanted device or system, the same or different wireless interface device 170 can be configured to use midfield-based transmissions to communicate with an implanted device.

In an example, the neuromodulation trial system 150 includes a magnet 168. The external stimulation controller 152 can include a switch, such as a magnetic switch or relay, that can be actuated by proximity (e.g., presence or absence) of the magnet 168. In an example, the switch can control one or more functions of the external stimulation controller 152. For example, the switch can be configured to disable or enable one or more functions of the external stimulation controller 152, such as delivery of neurostimulation signals to the patient.

The neuromodulation trial system 150 is thus configured to maximize patient comfort and case of use, and to provide reliable data on the effectiveness of neurostimulation for the individual. By allowing for a temporary and reversible trial of the therapy, the system helps determine the suitability of a permanent neurostimulation solution for managing the patient's symptoms or dysfunction.

In an example, various circuitry in the external stimulation controller 152 can be identically or similarly configured (i.e., via hardware or software) as circuitry in a permanent implantable device. Coherence between the designs of the neuromodulation trial system 150 and the permanent implantable device offers various advantages to the trial and the eventual long-term neurostimulation therapy.

First, the uniformity of the circuitry ensures that the electrical neurostimulation signals delivered during the trial phase are representative of those that can or would be provided by the permanent implantable device. Such consistency helps accurately evaluate the therapeutic benefits of a permanent system, and helps set appropriate expectations for the patient regarding the permanent therapy.

The identical, similar, or analogous circuitry between the trial and permanent systems and devices facilitates a seamless transition for patients who move forward with long-term implantation. The need for reprogramming or recalibration is minimized, thereby reducing the potential for patient discomfort and streamlining the process of converting trial success into sustained therapeutic relief. Furthermore, the shared circuitry allows for simultaneous software and/or firmware updates across both devices, ensuring that all patients benefit from the latest technological advancements. Maintenance protocols and quality control processes are also simplified, as the same procedures and components can be used for both the trial system and permanent devices.

From a development and manufacturing standpoint, the shared architecture between the external stimulation controller 152 and components of a permanent implantable device yields significant cost efficiencies. Research and development resources are optimized as the same technology platform is leveraged for both devices, which can also lead to a reduction in the overall cost of the therapy to the healthcare system and patients. Training of clinicians and patients is also simplified, as they need to become proficient with only one unified system. This can enhance the quality of patient care and increase the likelihood of positive clinical outcomes. Patients benefit from the continuity between the trial and permanent devices as well. For example, patient familiarity with the operation and sensation of the external stimulator can reduce anxiety and improve the overall patient experience when transitioning to the permanent implant.

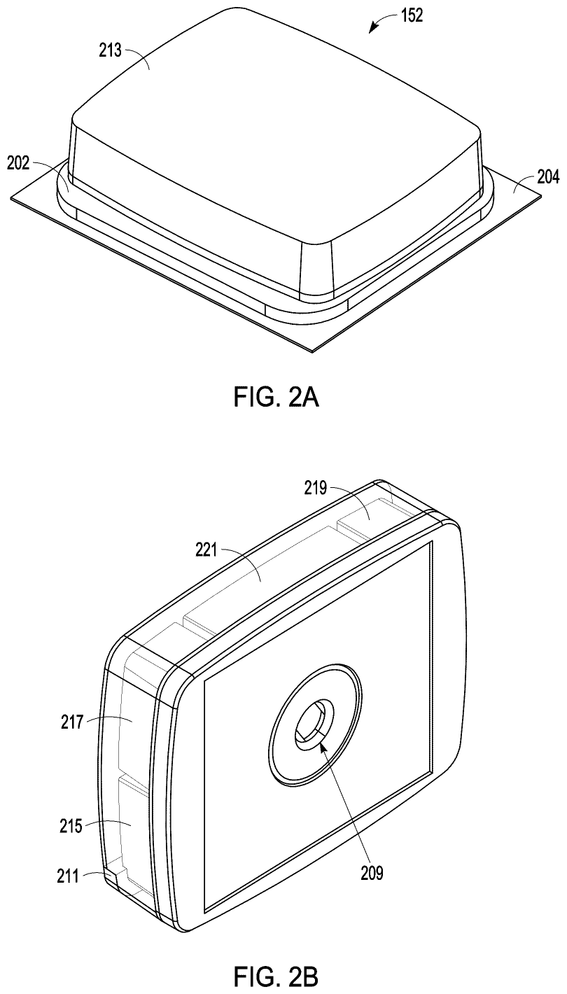

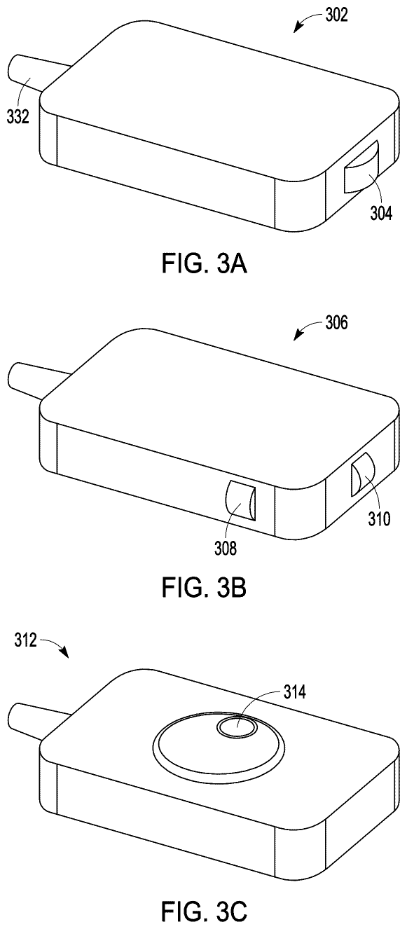

FIG. 2A and FIG. 2B illustrates generally perspective views of an example of the external stimulation controller 152. In an example, the external stimulation controller 152 includes a housing that is coupled to patient body tissue 204 using a ground pad 202. In an example, the housing includes one or more indicators, such as a display, a visual indicator 211 such as a light (e.g., an LED), or a haptic feedback device. Each indicator can be used to provide, for example, device status information or other feedback to the patient or clinician.

In the example of FIG. 2A, the ground pad 202 is interposed between a housing 213 of the external stimulation controller 152 and the body tissue 204. The ground pad 202 can optionally include a conductive adhesive that facilitates coupling between the ground pad 202 and the body tissue 204. The ground pad 202 provides a return path for the electrical current delivered by the stimulator to the neurostimulation lead or leads. This completes the electrical circuit, allowing the stimulation current to flow from the signal generator circuit, through the electrode(s) implanted near the target nerve(s), and back to the stimulator. In an example, the ground pad 202 provides a stable reference for the neurostimulation circuitry of the external stimulation controller 152, and helps to reduce electrical noise or interference that can affect performance of the stimulator or accuracy of the integrated physiologic sensors. Furthermore, the ground pad 202 can be configured to minimize skin irritation in the area where the external stimulation controller 152 is coupled to the patient body.

In an example, the external stimulation controller 152 comprises one or more physiologic sensors configured to measure physiologic status information about the patient. For example, the external stimulation controller 152 can include an ECG or EMG interface 209, such as can comprise one or more electrodes to sense electrical signals from the patient. In an example, the external stimulation controller 152 can include one or more other sensors such as an accelerometer configured to sense patient motion, posture, or activity, or a thermometer configured to measure patient skin temperature, or an oximeter, or a galvanic skin response (GSR) sensor, or a glucose sensor, among other sensors. Incorporating such sensors into the external stimulation controller 152 allows for real-time monitoring of a physiologic status of the patient. The external stimulation controller 152 can be configured to use the sensor information to titrate or adjust therapy, or can report such sensor information to the patient or clinician using the patient device 166 or clinician device 164. That is, the external stimulation controller 152 can be configured to automatically adjust the neurostimulation signals for a neurostimulation therapy based on real-time data received from the one or more sensors.

The external stimulation controller 152 can include, among other things, a processor circuit 215, a signal generator circuit 217, a transceiver circuit 219, and a battery 221. In an example, the signal generator circuit 217 is coupled to the processor circuit 215. The signal generator circuit 217 can be configured to generate neurostimulation signals in response to instructions from the processor circuit 215. The transceiver circuit 219 can be coupled to the processor circuit 215. In an example, the transceiver circuit 219 is configured to interface with, or receive instructions from, the wireless interface device 170. The instructions from the wireless interface device 170 can include, for example, instructions to initiate, adjust, or terminate a particular therapy. In an example, the instructions from the wireless interface device 170 include therapy parameters, such as signal timing or morphology parameters.

In an example, the external stimulation controller 152 can comprise a lead interface (not shown in FIG. 2A or FIG. 2B). The lead interface can be configured to couple with a lead, and the lead can be at least partially implantable. Therapy signals from the signal generator circuit 217 can be provided to the lead using the lead interface.

In an example, the external stimulation controller 152 can include or use a magnetic switch (not shown in FIG. 2A or FIG. 2B). At least one function or operation of the external stimulation controller 152 can be changed based on a state of the magnetic switch. For example, in the presence of a magnet, the magnetic switch can be closed. When the magnetic switch is closed, for example, a circuit can be completed to transmit power from the battery 221 to the signal generator circuit 217 or to one or more other components of the external stimulation controller 152. When the magnetic switch is open, the circuit can be interrupted and, accordingly, the signal generator circuit 217 can be disabled. Other components or operations of the external stimulation controller 152 can similarly be controlled by the magnetic switch.

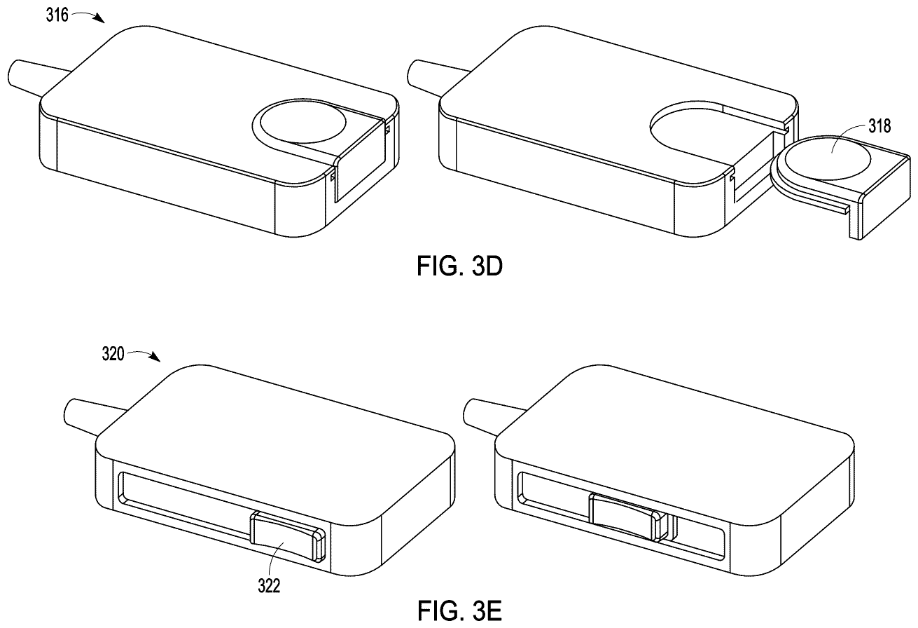

FIG. 3A through FIG. 3G illustrate generally various examples of the housing of the external stimulation controller 152. Some of the examples include or use a switch or button, one or more of which can be implemented at least in part using the magnet 168. In some examples, the magnet 168 is physically removable from the housing. In some examples, the magnet 168 is configured to slide or rotate toward or away from a switch inside the housing, but the magnet 168 remains coupled to the housing at a fulcrum or axis. In an example, the switch is configured to be actuated without using a magnet. A switch that is actuated by a magnet can be advantageous, for example, because the housing that encloses the switch can be hermetic or water-tight. The various switches, buttons, or other interface features shown in FIG. 3A through FIG. 3G can be used to enable or disable one or more functions of the external stimulation controller 152, such as to enable or disable therapy or electrostimulation.

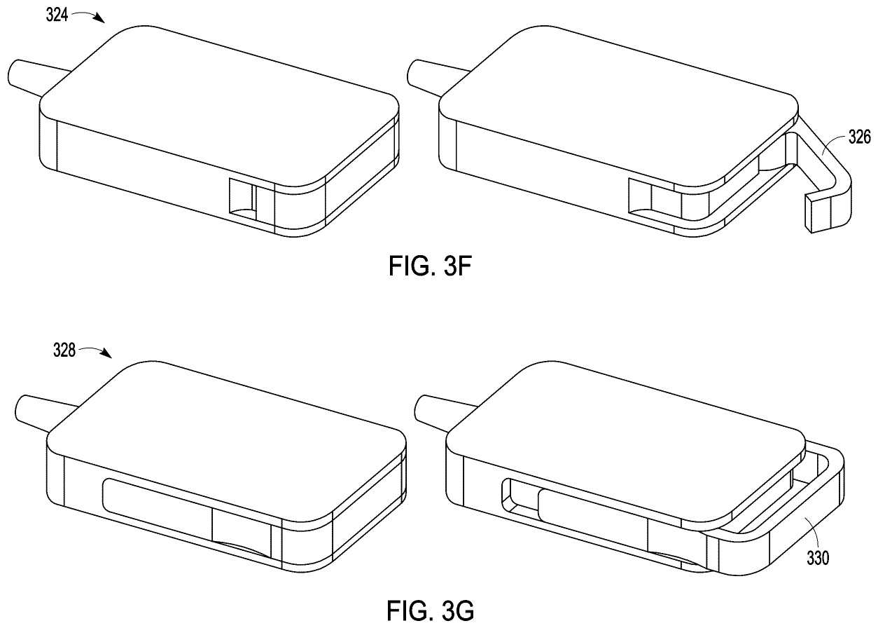

FIG. 3A shows a first example 302 of a housing of the external stimulation controller 152 with a first switch or first button 304. The first button 304 can be disposed on a side edge of the housing to help minimize unintentional actuation. The first button 304 can be a pushbutton that is configured to enable or disable one or more functions of the external stimulation controller 152. In the example of FIG. 3A, the first example 302 includes a trial lead interface 332 that is configured to couple with a trial lead (see, e.g., the trial lead assembly 452 discussed in the example of FIG. 4).

FIG. 3B shows a second example 306 of a housing of the external stimulation controller 152 with a first side button 308 and a second side button 310. The first side button 308 and second side button 310 can be provided on adjacent or opposite side edges of the housing. In an example, the first side button 308 and the second side button 310 can be actuated together to enable or disable one or more functions of the external stimulation controller 152. Requiring concurrent actuation of the two buttons together can help reduce occurrences of unintentional actuation. In other examples, the first side button 308 and the second side button 310 can be configured to independently control respective different functions of the external stimulation controller 152.

FIG. 3C shows a third example 312 of a housing of the external stimulation controller 152 with a circular pushbutton or rotatable interface 314. The rotatable interface 314 can have two or more positions to control respective functions of the external stimulation controller 152.

FIG. 3D shows a fourth example 316 of a housing of the external stimulation controller 152 with a removable magnetic switch 318. In an example, the removable magnetic switch 318 comprises the magnet 168. At left in FIG. 3D, the removable magnetic switch 318 is mated with a receptacle on a surface of the housing. At right, the removable magnetic switch 318 is separated from the receptacle, for example, to disengage therapy provided by the external stimulation controller 152.

FIG. 3E shows a fifth example 320 of a housing of the external stimulation controller 152 with a first sliding switch 322. At left in FIG. 3E, the first sliding switch 322 is in a first position, and at right in FIG. 3E, the first sliding switch 322 is in a second position. The different positions of the first sliding switch 322 can correspond to respective different operating modes of the external stimulation controller 152.

FIG. 3F shows a sixth example 324 of a housing of the external stimulation controller 152 with a lever switch 326. At left in FIG. 3F, the lever switch 326 is in a first position, mated against a sidewall of the housing. At right in FIG. 3F, a body of the lever switch 326 is in a second position rotated away from the sidewall of the housing. The body of the lever switch 326 can comprise the magnet 168. The lever switch 326 is coupled to the housing at one end to help ensure the switch is not fully removed.

FIG. 3G shows a seventh example 328 of a housing of the external stimulation controller 152 with a second sliding switch 330. At left in FIG. 3G, the second sliding switch 330 is in a first position, mated against a sidewall of the housing. At right in FIG. 3G, the second sliding switch 330 is in a second position where at least a portion of the second sliding switch 330 is spaced apart from the sidewall of the housing. The spaced apart portion of the second sliding switch 330 can comprise the magnet 168.

FIG. 4 illustrates generally an example of a trial lead assembly 452, such as can comprise the first trial lead 156 or the second trial lead 158. The trial lead assembly 452 can comprise a coiled conductor. The coiled configuration helps maximize flexibility of the lead, which in turn helps ensure chronic placement of the lead and its electrode during the trial period. In an example, the lead comprises a single conductor and thus a single electrode for delivery of neurostimulation signals. In other examples, a trial lead can comprise multiple conductors and respective electrodes.

In an example, the lead conductor comprises 316 LVM stainless steel with a PFA coating. The conductor can have various dimensions. For example, an outer diameter of the coiled conductor can be less than or equal to about 0.035 inches. The coiled conductor can wound about a core (e.g., during fabrication, and the core can be removable). The core can be an air core or can comprise a biocompatible material. In an example, the core has a diameter of about 0.014 inches or less. In an example, the core comprises a void region and a rigid stylet can be inserted into the void region of the coiled conductor to facilitate conductor insertion into patient tissue. The stylet can be removed after the conductor is implanted in the patient.

The coiled lead can be coated with a biocompatible insulator, and the lead ends (or one or more other portions of the lead) can be ablated. For example, an ablated distal end can comprise an electrode area and can be terminated using, e.g., a blunted feature 454, such as can be conductive or non-conductive. An ablated proximal end can comprise a pin 456, such as can be coupled to a header of the external stimulation controller 152 to interface with the signal generator circuitry therein.

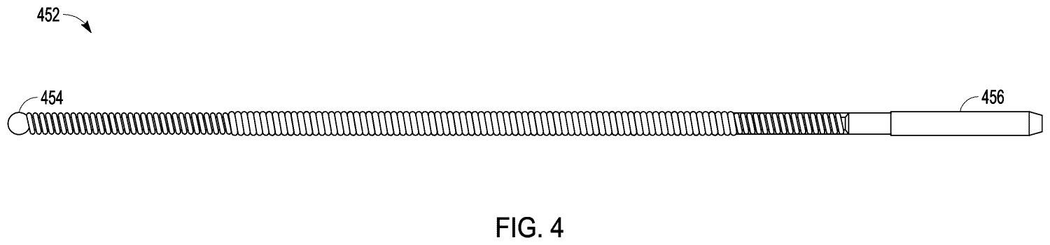

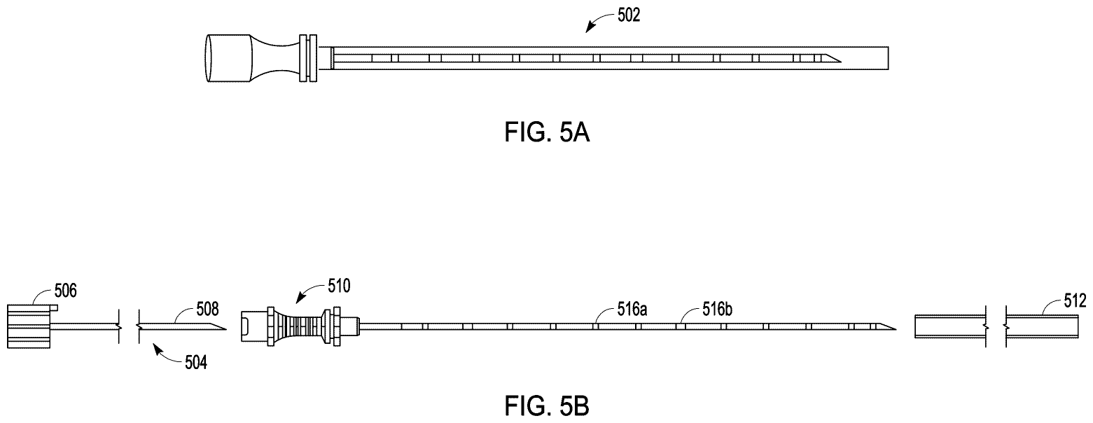

FIG. 5A illustrates generally a perspective view of an example of a foramen needle assembly 502. The foramen needle assembly 502 can be used to implant a neurostimulation lead, such as the first trial lead 156 or the second trial lead 158.

FIG. 5B illustrates generally an exploded view of the example foramen needle assembly 502. In an example, the foramen needle assembly 502 includes a stylet assembly 504, a needle assembly 510, and a protective sheath 512. The stylet assembly 504 includes a luer cap 506 coupled to a stylet 508. The needle assembly 510 includes a proximal portion configured to couple with the luer cap 506, and a distal needle with one or more depth markers, such as can be visible under fluoroscopy. The example illustration of FIG. 5B shows several depth markers, including a first depth marker 516a and a second depth marker 516b. The needle portion of the needle assembly 510 can be coated with Parylene-C, for example except for the distal tip and proximal end. In an example, the depth markers can be chemically etched in the needle portion. The stylet 508 is configured to fit inside the needle of the needle assembly 510, and the needle of the needle assembly 510 is configured to fit inside the protective sheath 512.

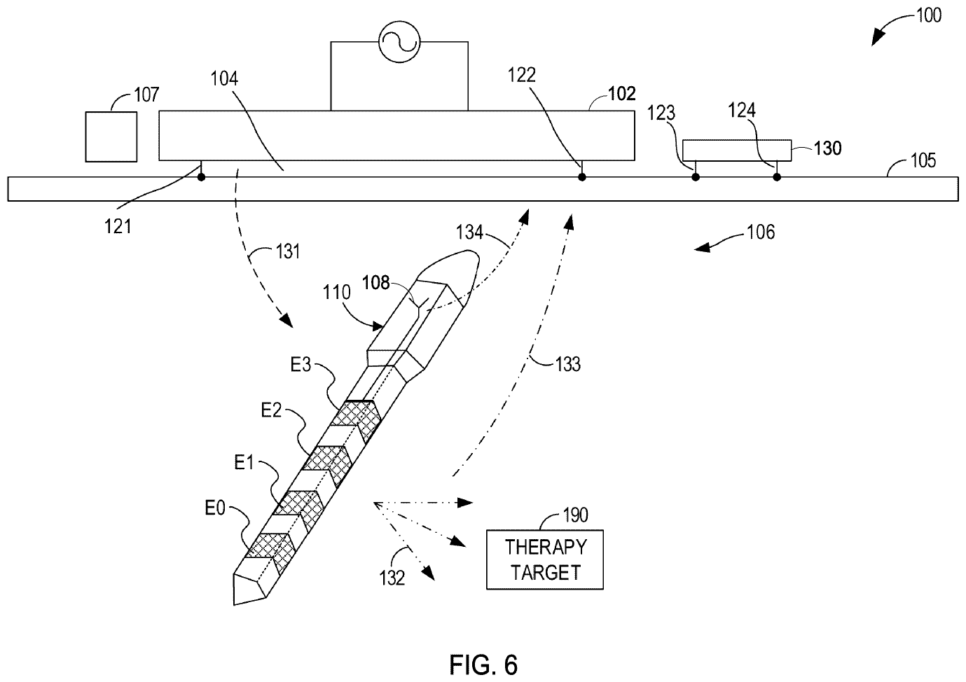

FIG. 6 illustrates generally a schematic of an embodiment of a system 100 that includes a permanent implanted neurostimulation device. The system 100 includes an example of an external source 102, such as a midfield transmitter source, sometimes referred to as a midfield coupler or external unit or external power unit. The external source 102 can be located at or above an interface 105 between air 104 and a higher-index material 106, such as body tissue. The external source 102 can produce a source current (e.g., an in-plane source current). The source current can generate an electric field and a magnetic field. The magnetic field can include a non-negligible component that is parallel to the surface of the source 102 and/or to a surface of the higher-index material 106 (e.g., a surface of the higher-index material 106 that faces the external source 102). In accordance with several embodiments, the external source 102 may comprise structural features and functions described in connection with the midfield couplers and external sources included in WIPO Publication No. WO/2015/179225 published on Nov. 26, 2015 and titled “MIDFIELD COUPLER”, which is incorporated herein by reference in its entirety.

In an example, the external source 102 comprises the wireless interface device 170 from the example of the neuromodulation trial system 150. The external source 102 can be configured for power and/or data communication with the external stimulation controller 152 when the external source 102 (or wireless interface device 170) is in a trial therapy mode. In the trial therapy mode, power transmission circuitry of the external source 102 (or wireless interface device 170) can be disabled or deactivated. In another therapy mode, the external source 102 (or wireless interface device 170) can be configured to enable the power transmission circuitry to transmit power to one or more implanted devices. That is, the external source 102 can be configured for power and/or data communication with an implantable device 110. In some examples, power and/or data signal receive circuitry or other components can be substantially the same or identical in respective instances of the external stimulation controller 152 and the external source 102.

In an example, the external source 102 can include at least a pair of outwardly facing electrodes 121 and 122. The electrodes 121 and 122 can be configured to contact a tissue surface, for example, at the interface 105. In one or more examples, the external source 102 is configured for use with a sleeve, pocket, or other garment or accessory that maintains the external source 102 adjacent to the higher-index material 106, and that optionally maintains the electrodes 121 and 122 in physical contact with a tissue surface. In one or more examples, the sleeve, pocket, or other garment or accessory can include or use a conductive fiber or fabric, and the electrodes 121 and 122 can be in physical contact with the tissue surface via the conductive fiber or fabric. In an example, the ECG or EMG interface 209 can comprise at least one of the electrodes 121 and 122.

In one or more examples, more than two outwardly facing electrodes can be used and processor circuitry on-board or auxiliary to the source 102 can be configured to select an optimal pair or group of electrodes to use to sense farfield signal information (e.g., signal information corresponding to a delivered therapy signal or to a nearfield signal). In such embodiments, the electrodes can operate as antennas. In one or more examples, the source 102 includes three outwardly facing electrodes arranged as a triangle, or four outwardly facing electrodes arranged as a rectangle, and any two or more of the electrodes can be selected for sensing and/or can be electrically grouped or coupled together for sensing or diagnostics. In one or more examples, the processor circuitry can be configured to test multiple different electrode combination selections to identify an optimal configuration for sensing a farfield signal.

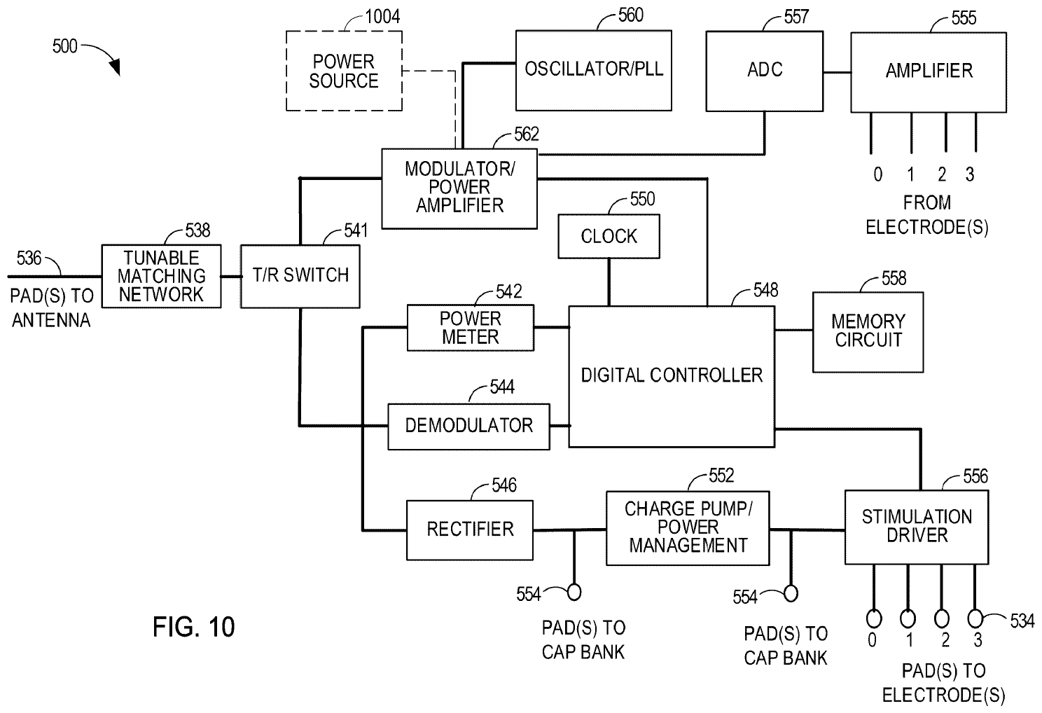

FIG. 6 illustrates an embodiment of an implantable device 110 (e.g., a permanent device, as contrasted with a temporary trial device), such as can include a multi-polar therapy delivery device configured to be implanted in the higher-index material 106 or in a blood vessel. In one or more examples, the implantable device 110 includes all or a portion of the circuitry 500 from FIG. 10, discussed in further detail below. In one or more examples, the implantable device 110 is implanted in tissue below the tissue-air interface 105. In FIG. 6, the implantable device 110 includes an elongate body and multiple electrodes E0, E1, E2, and E3 that are axially spaced apart along a portion of the elongate body. In an example, the trial lead assembly 452 can comprise multiple electrodes that correspond to one or more of the E0, E1, E2, and/or E3 electrodes of the permanent implantable device 110. That is, one or more electrodes of the trial lead assembly 452 can have substantially the same size, shape, orientation, spacing, or other similar characteristic as one or more corresponding electrodes of the permanent device. The implantable device 110 includes receiver and/or transmitter circuitry that can enable communication between the implantable device 110 and the external source 102.

The various electrodes E0-E3 can be configured to deliver electrostimulation therapy to patient tissue, such as at or near a neural or muscle target. In one or more examples, at least one electrode can be selected for use as an anode and at least one other electrode can be selected for use as a cathode to define an electrostimulation vector. In one or more examples, electrode E1 is selected for use as an anode and electrode E2 is selected for use as a cathode. Together, the E1-E2 combination defines an electrostimulation vector V12. Various vectors can be configured independently to provide a neural electrostimulation therapy to the same or different tissue target, such as concurrently or at different times.

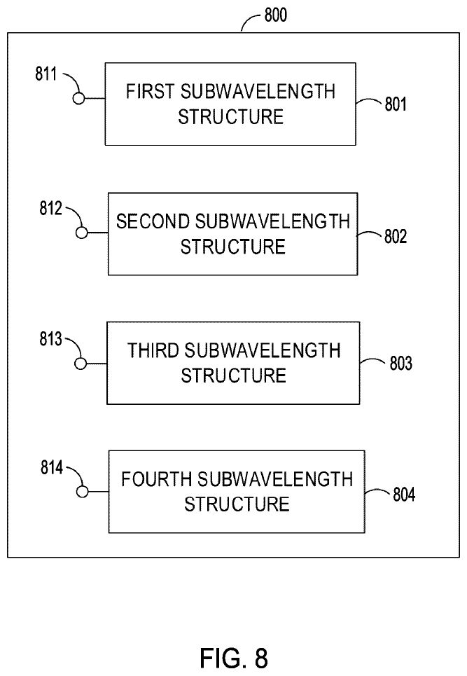

In one or more examples, the source 102 includes an antenna (see, e.g., FIG. 8) and the implantable device 110 includes an antenna 108 (e.g., and electric field-based or magnetic field-based antenna). The antennas can be configured (e.g., in length, width, shape, material, etc.) to transmit and receive signals at substantially the same frequency. The implantable device 110 can be configured to transmit power and/or data signals through the antenna 108 to the external source 102 and can receive power and/or data signals transmitted by the external source 102. The external source 102 and implantable device 110 can be used for transmission and/or reception of RF signals. A transmit/receive (T/R) switch can be used to switch each RF port of the external source 102 from a transmit (transmit data or power) mode to a receive (receive data) mode. A T/R switch can similarly be used to switch the implantable device 110 between transmit and receive modes.

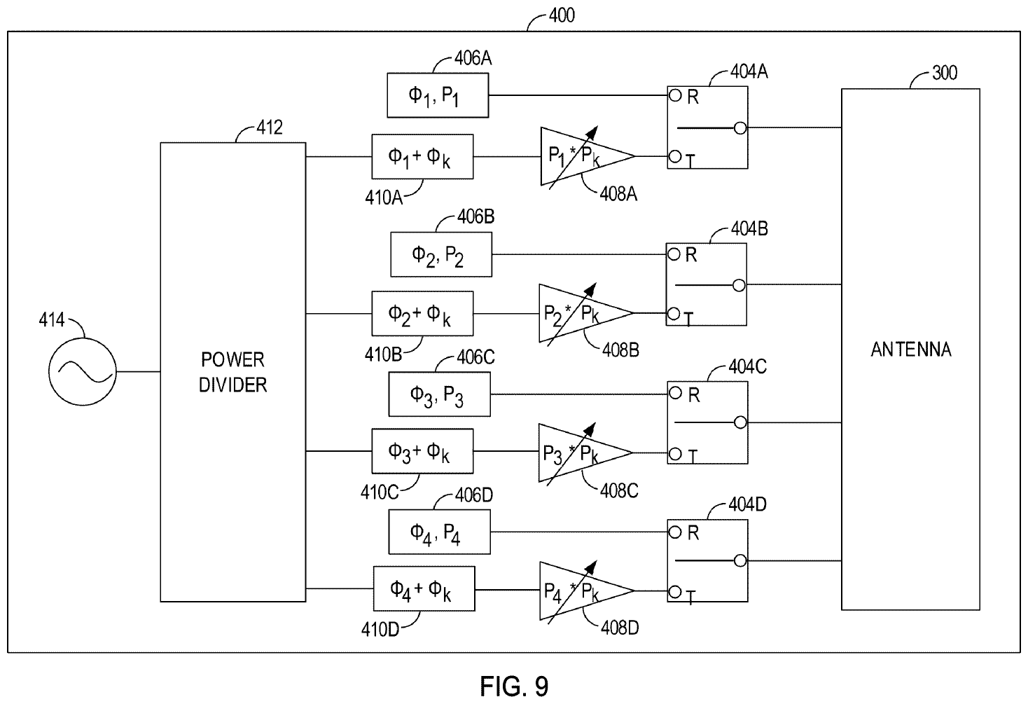

In one or more examples, a receive terminal on the external source 102 can be connected to one or more components that detect a phase and/or amplitude of a received signal from the implantable device 110. The phase and amplitude information can be used to program a phase of the transmit signal, such as to be substantially the same relative phase as a signal received from the implantable device 110. To help achieve this, the external source 102 can include or use a phase-matching and/or amplitude-matching network, such as shown in the embodiment of FIG. 9. The phase-matching and/or amplitude matching network can be configured for use with a midfield antenna that includes multiple ports, such as shown in the embodiment of FIG. 8.

Referring again to FIG. 6, in one or more examples, the implantable device 110 can be configured to receive a midfield signal 131 from the external source 102. The midfield signal 131 can include power and/or data signal components. In some embodiments, a power signal component can include one or more data components embedded therein. In one or more examples, the midfield signal 131 includes configuration data for use by the implantable device 110. The configuration data can define, among other things, therapy signal parameters, such as a therapy signal frequency, pulse width, amplitude, or other signal waveform parameters.

In one or more examples, the implantable device 110 can be configured to deliver an electrostimulation therapy to a therapy target 190, such as can include a neural target (e.g., a nerve, or other tissue such as a vein, connective tissue, or other tissue that includes one or more neurons within or near the tissue), a muscle target, or other tissue target. An electrostimulation therapy delivered to the therapy target 190 can be provided using a portion of a power signal received from the external source 102. Examples of the therapy target 190 can include nerve tissue or neural targets, for example including nerve tissue or neural targets at or near cervical, thoracic, lumbar, or sacral regions of the spine, brain tissue, muscle tissue, abnormal tissue (e.g., tumor or cancerous tissue), targets corresponding to sympathetic or parasympathetic nerve systems, targets at or near peripheral nerve bundles or fibers, at or near other targets selected to treat incontinence, urinary urge, overactive bladder, fecal incontinence, constipation, pain, neuralgia, pelvic pain, movement disorders or other diseases or disorders, deep brain stimulation (DBS) therapy targets or any other condition, disease or disorder (such as those other conditions, diseases, or disorders identified herein).

Delivering the electrostimulation therapy can include using a portion of a power signal received via the midfield signal 131, and providing a current signal to an electrode or an electrode pair (e.g., two or more of E0-E3), coupled to the implantable device 110, to stimulate the therapy target 190. As a result of the current signal provided to the electrode(s), a nearfield signal 132 can be generated. An electric potential difference resulting from the nearfield signal 132 can be detected remotely from the therapy delivery location. Various factors can influence where and whether the potential difference can be detected, including, among other things, characteristics of the therapy signal, a type or arrangement of the therapy delivery electrodes, and characteristics of any surrounding biologic tissue. Such a remotely detected electric potential difference can be considered a farfield signal 133. The farfield signal 133 can represent an attenuated portion of the nearfield signal 132. That is, the nearfield signal 132 and the farfield signal 133 can originate from the same signal or field, such as with the nearfield signal 132 considered to be associated with a region at or near the implantable device 110 and the therapy target 190, and with the farfield signal 133 considered to be associated with other regions more distal from the implantable device 110 and the therapy target 190. In one or more examples, information about the implantable device 110, or about a previously-provided or future planned therapy provided by the implantable device 110, can be encoded in a therapy signal and detected and decoded by the external source 102 by way of the farfield signal 133.

In one or more examples, the implantable device 110 can be configured to provide a series of electrostimulation pulses to a tissue target (e.g., a neural target). For example, the implantable device 110 can provide multiple electrostimulation pulses separated in time, such as using the same or different electrostimulation vectors, to provide a therapy. In one or more examples, a therapy comprising multiple signals can be provided to multiple different vectors in parallel, or can be provided in sequence such as to provide a series or sequence of electrostimulation pulses to the same neural target. Thus, even if one vector is more optimal than the others for eliciting a patient response, the therapy as a whole can be more effective than stimulating only the known-optimal vector because (1) the target may experience a rest period during periods of non-stimulation, and/or (2) stimulating the areas nearby and/or adjacent to the optimal target can elicit some patient benefit.

The system 100 or neuromodulation trial system 150 can include a sensor 107 at or near the interface 105 between air 104 and the higher-index material 106. The sensor 107 can include, among other things, one or more electrodes, an optical sensor, an accelerometer, a temperature sensor, a force sensor, a pressure sensor, or a surface electromyography (EMG) device. The sensor 107 may comprise multiple sensors (e.g., two, three, four or more than four sensors). Depending on the type of sensor(s) used, the sensor 107 can be configured to monitor electrical, muscle, or other activity near the device 110 and/or near the source 102. For example, the sensor 107 can be configured to monitor muscle activity at a tissue surface. If muscle activity greater than a specified threshold activity level is detected, then a power level of the source 102 and/or of the device 110 can be adjusted. In one or more examples, the sensor 107 can be coupled to or integrated with the source 102 or the wireless interface device 170, and in other examples, the sensor 107 can be separate from, and in data communication with (e.g., using a wired or wireless electrical coupling or connection), the source 102 and/or the device 110.

The system 100 or the neuromodulation trial system 150 can include a farfield sensor device 130 that can be separate from, or communicatively coupled with, one or more of the source 102 or the wireless interface device 170 and the sensor 107. The farfield sensor device 130 can include two or more electrodes and can be configured to sense a farfield signal, such as the farfield signal 133 corresponding to a therapy delivered by the device 110. The farfield sensor device 130 can include at least one pair of outwardly facing electrodes 123 and 124 configured to contact a tissue surface, for example, at the interface 105. In one or more examples, three or more electrodes can be used, and processor circuitry on-board or auxiliary to the farfield sensor device 130 can select various combinations of two or more of the electrodes for use in sensing the farfield signal 133. In one or more examples, the farfield sensor device 130 can be configured for use with a sleeve, pocket, or other garment or accessory that maintains the farfield sensor device 130 adjacent to the higher-index material 106, and that optionally maintains the electrodes 123 and 124 in physical contact with a tissue surface. In one or more examples, the sleeve, pocket, or other garment or accessory can include or use a conductive fiber or fabric, and the electrodes 123 and 124 can be in physical contact with the tissue surface via the conductive fiber or fabric.

In one or more examples, the external source 102 provides a midfield signal 131 including power and/or data signals to the implantable device 110. The midfield signal 131 includes a signal (e.g., an RF signal) having various or adjustable amplitude, frequency, phase, and/or other signal characteristics. The implantable device 110 can include an antenna, such as described below, that can receive the midfield signal 131 and, based on characteristics of receiver circuitry in the implantable device 110, can modulate the received signal at the antenna to thereby generate a backscatter signal. In one or more examples, the implantable device 110 can encode information in the backscatter signal 112, such as information about a characteristic of the implantable device 110 itself, about a received portion of the midfield signal 131, about a therapy provided by the implantable device 110, and/or other information. The backscatter signal 112 can be received by an antenna at the external source 102 and/or the farfield sensor device 130, or can be received by another device. In one or more examples, a biological signal can be sensed by a sensor of the implantable device 110, such as a glucose sensor, an electropotential (e.g., an electromyography sensor, electrocardiogramansor, resistance, or other electrical sensor), a light sensor, a temperature, a pressure sensor, an oxygen sensor, a motion sensor, or the like. A signal representative of the detected biological signal can be modulated onto the backscatter signal 112. Other sensors are discussed elsewhere herein. In such embodiments, the sensor 107 can include a corresponding monitor device, such as a glucose, temperature, ECG, EMG, oxygen, or other monitor, such as to receive, demodulate, interpret, and/or store data modulated onto the backscatter signal.

In one or more examples, the external source 102 and/or the implantable device 110 can include an optical transceiver configured to facilitate communication between the external source 102 and the implantable device 110. The external source 102 can include a light source, such as a photo laser diode or LED, or can include a photo detector, or can include both of a light source and a photo detector. The implantable device 110 can include a light source, such as a photo laser diode or LED, or can include a photo detector, or can include both of a light source and a photo detector. In an example, the external source 102 and/or implantable device 110 can include a window, such as made of quartz, glass, or other translucent material, adjacent to its light source or photo detector.

In an example, optical communications can be separate from or supplemental to an electromagnetic coupling between the external source 102 and the implantable device 110. Optical communication can be provided using light pulses modulated according to various protocols, such as using pulse position modulation (PPM). In an example, a light source and/or photo detector on-board the implantable device 110 can be powered by a power signal received at least in part via midfield coupling with the external source 102.

In an example, a light source at the external source 102 can send a communication signal through skin, into subcutaneous tissue, and through an optical window (e.g., quartz window) in the implantable device 110. The communication signal can be received at a photo detector on-board the implantable device 110. Various measurement information, therapy information, or other information from or about the implantable device can be encoded and transmitted from the implantable device 110 using a light source provided at the implantable device 110. The light signal emitted from the implantable device 110 can travel through the same optical window, subcutaneous tissue, and skin tissue, and can be received at photo detector on-board the external source 102. In an example, the light sources and/or photo detectors can be configured to emit and/or receive, respectively, electromagnetic waves in the visible or infrared ranges, such as in a range of about 670-910 nm wavelength (e.g., 670 nm-800 nm, 700 nm-760 nm, 670 nm-870 nm, 740 nm-850 nm, 800 nm-910 nm, overlapping ranges thereof, or any value within the recited ranges).

In an example, the external source 102 can include various circuitry to facilitate device reset, storage, user access, and other features. For example, the external source 102 can include a latching switch to provide a device-level power switch, such as can be used to remove power from drive or sense circuitry provided in the external source 102. In an example, the external source 102 can include a reed switch (e.g., a magnetic reed switch) that can be activated to perform a manual reset or to enter a device configuration mode or learning mode. In an example, the external source 102 can include an environmental sensor (e.g., a thermistor, humidity or moisture sensor, etc.) to detect device conditions and change device operating behavior accordingly. For example, information from a thermistor can be used to indicate a fault condition to prevent device overheating.

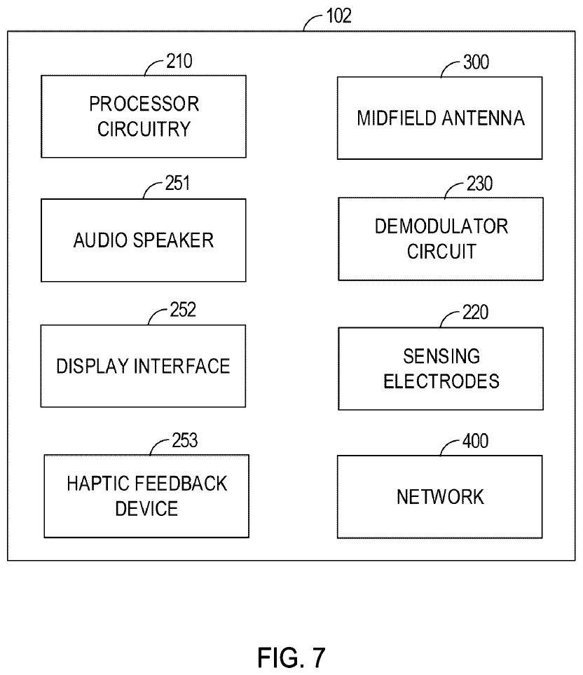

FIG. 7 illustrates, by way of example, a block diagram of an embodiment of a midfield source device, such as the external source 102. In an example, the wireless interface device 170 or the external stimulation controller 152 of the neuromodulation trial system 150 comprises some or all of the same components used in the external source 102.

The external source 102 can include various components, circuitry, or functional elements that are in data communication with one another. In the example of FIG. 7, the external source 102 includes components, such as processor circuitry 210, one or more sensing electrodes 220 (e.g., including the electrodes 121 and 122), a demodulator circuitry 230, a phase-matching or amplitude-matching network 400, a midfield antenna 300, and/or one or more feedback devices, such as can include or use an audio speaker 251, a display interface 252, and/or a haptic feedback device 253. The processor circuitry 210 can be configured to coordinate the various functions and activities of the components, circuitry, and/or functional elements of the external source 102.

The midfield antenna 300 can be configured to provide a midfield excitation signal, such as can include RF signals having a non-negligible H-field component that is substantially parallel to an external tissue surface. In one or more examples, the RF signals can be adapted or selected to manipulate an evanescent field at or near a tissue surface, such as to transmit a power and/or data signal to respective different target devices (e.g., the implantable device 110, or any one or more other implantable devices discussed herein) implanted in tissue. The midfield antenna 300 can be further configured to receive backscatter or other wireless signal information that can be demodulated by the demodulator circuitry 230. The demodulated signals can be interpreted by the processor circuitry 210.

The midfield antenna 300 can include a dipole antenna, a loop antenna, a coil antenna, a slot or strip antenna, or other antenna. The antenna 300 can be shaped and sized to receive signals in a range of between about 400 MHz and about 4 GHz (e.g., between 400 MHz and 1 GHz, between 400 MHz and 3 GHz, between 500 MHz and 2 GHz, between 1 GHz and 3 GHz, between 500 MHz and 1.5 GHz, between 1 GHz and 2 GHz, between 2 GHz and 3 GHz, overlapping ranges thereof, or any value within the recited ranges). For embodiments incorporating a dipole antenna, the midfield antenna 300 may comprise a straight dipole with two substantially straight conductors, a folded dipole, a short dipole, a cage dipole, a bow-tic dipole or batwing dipole.

The demodulator circuitry 230 can be coupled to the sensing electrodes 220. In one or more examples, the sensing electrodes 220 can be configured to receive the farfield signal 133, such as based on a therapy provided by the implantable device 110, such as can be delivered to the therapy target 190. The therapy can include an embedded or intermittent data signal component that can be extracted from the farfield signal 133 by the demodulator circuitry 230. For example, the data signal component can include an amplitude-modulated or phase-modulated signal component that can be discerned from background noise or other signals and processed by the demodulator circuitry 230 to yield an information signal that can be interpreted by the processor circuitry 210. Based on the content of the information signal, the processor circuitry 210 can instruct one of the feedback devices to alert a patient, caregiver, or other system or individual. For example, in response to the information signal indicating successful delivery of a specified therapy, the processor circuitry 210 can instruct the audio speaker 251 to provide audible feedback to a patient, can instruct the display interface 252 to provide visual or graphical information to a patient, and/or can instruct the haptic feedback device 253 to provide a haptic stimulus to a patient. In one or more examples, the haptic feedback device 253 includes a transducer configured to vibrate or to provide another mechanical signal.

In an example, the external source 102 includes a wireless communication circuit configured to communicate data with an external patient or clinician interface device, and/or to communicate data with the external stimulation controller 152. The external device, such as can include the clinician device 164 and/or the patient device 166, can include an interface that allows the patient or clinician to interact with the external source 102 (e.g., the external stimulation controller 152) or implantable device 110, such as to update or adjust one or more therapy settings.

FIG. 8 illustrates generally a schematic view of an embodiment of a midfield antenna 800 with multiple excitable structures, including subwavelength structures 801, 802, 803, and 804. The midfield antenna 800 can comprise a portion of the wireless interface device 170 or the external source 102. The midfield antenna 800 can include a midfield plate structure with a substantially planar surface. The one or more subwavelength structures 801-804 can be formed in the plate structure. In the example of FIG. 8, the antenna 800 includes a first subwavelength structure 801, a second subwavelength structure 802, a third subwavelength structure 803, and a fourth subwavelength structure 804. Fewer or additional subwavelength structures can be used. The subwavelength structures can be excited individually or selectively by one or more RF ports (e.g., first through fourth RF ports 811, 812, 813, and 814) respectively coupled thereto.

A “subwavelength structure” can include a hardware structure with dimensions defined relative to a wavelength of a field that is rendered and/or received using the midfield antenna 800. For example, for a given λ₀ corresponding to a signal wavelength in air, a source structure that includes one or more dimensions less than λ₀ can be considered to be a subwavelength structure. Various designs or configurations of subwavelength structures can be used. Some examples of a subwavelength structure can include a slot in a planar structure, or a strip or patch of a conductive sheet of substantially planar material. Various examples of midfield antenna and excitable structures are discussed elsewhere herein. In some examples, the excitable structures include or use striplines or microstrips.

In an example, the midfield antenna 800 and its associated drive circuitry (discussed elsewhere herein) are configured to provide signals to manipulate or influence an evanescent field at or adjacent to tissue, where tissue serves as a medium with a relatively high dielectric constant (e.g., tissue is a high-κ medium). That is, energy from the antenna 800 can be directed through the tissue or other high-κ medium rather than through air. An efficiency of transmission from the midfield antenna 800 can be greatest when the antenna 800 is properly loaded by tissue, and the efficiency can be intentionally low when unloaded by tissue.

FIG. 9 illustrates generally a phase-adjusting/matching or amplitude-adjusting/matching network 400. In an example, the network 400 can include the antenna 300, and the antenna 300 can be electrically coupled to a plurality of switches 404A, 404B, 404C, and 404D, for example, via the first through fourth RF ports 811, 812, 813, and 814 illustrated in FIG. 8. The switches 404A-D are each electrically coupled to a respective phase and/or amplitude detector 406A, 406B, 406C, and 406D, and a respective variable gain amplifier 408A, 408B, 408C, and 408D. Each amplifier 408A-D is electrically coupled to a respective phase shifter 410A, 410B, 410C, and 410D, and each phase shifter 410A-D is electrically coupled to a source, such as can include a power divider 412 that receives an input signal 414 to be transmitted using the external source 102 or the wireless interface device 170. In an example, the phase shifters 410A-D are digitally-configurable devices configured to adjust a phase response in specified intervals. For example, the phase shifters 410A-D can comprise 4-bit phase control devices that can be configured to adjust phase in increments of 22.5 degrees.

In one or more examples, the switches 404A-D can be configured to select either a receive line (“R”) or a transmit line (“T”). A number of switches 404A-D of the network 400 can be equal to a number of ports of a midfield source such as the antenna 300. In the example of the network 400, the midfield source includes four ports (e.g., corresponding to the four subwavelength structures in the antenna 300 of the example of FIG. 8), however any number of ports (and switches), such as one, two, three, four, five, six, seven, eight or more, can be used.

The phase and/or amplitude detectors 406A-D are configured to detect a phase (Φ1, Φ2, Φ3, Φ4) and/or power (P1, P2, P3, P4) of a signal received at each respective port of the midfield source. In one or more examples, the phase and/or amplitude detectors 406A-D can be implemented in one or more modules (hardware modules that can include electric or electronic components arranged to perform an operation, such as determining a phase or amplitude of a signal), such as including a phase detector module and/or an amplitude detector module. The detectors 406A-D can include analog and/or digital components arranged to produce one or more signals representative of a phase and/or amplitude of a signal received at the external source 102 or the wireless interface device 170.

The amplifiers 408A-D can receive respective inputs from the phase shifters 410A-D (e.g., Pk phase shifted by Φk, Φ1+Φk, @2+Φk, Φ3+Φk, or Φ4+Φk). The output of the amplifier, O, is generally the output of the power divider, M, when the input signal 414 has an amplitude of 4*M (in the embodiment of FIG. 9), multiplied by the gain of the amplifier Pi*Pk. Pk can be set dynamically as the values for P1, P2, P3, and/or P4 change. Φk can optionally be a constant or a variable.

In one or more examples, the phase shifters 410A-D can dynamically or responsively configure the relative phases of the signals provided to the ports. That is, a polarization of the external wireless interface device 170 or external source 102 can be adjusted to optimize power communication to another device, such as by optimizing constructive interference of electric field and magnetic field components, and by minimizing destructive interference of the same. In an example, one or more of the phase shifters 410A-410D can be adjusted based on information received from the implantable device or from a patient, as further discussed below. In an example, one or more of the phase shifters 410A-D can be adjusted based on amplitude or phase information received from the detectors 406A-D.

In an example, excitation signal paths in the network 400 can be configured to operate in pairs. A first pair of signal paths can include, for example, the signal path corresponding to the first phase shifter 410A and the signal path corresponding to the third phase shifter 410C. Similarly, a second pair of signal paths can include, for example, the signal path corresponding to the second phase shifter 410B and the signal path corresponding to the fourth phase shifter 410D. In an example, each of the phase shifters drives a respective transmitter antenna port, as further discussed elsewhere herein. By way of example, each of the phase shifters can provide a signal to a respective excitable structure (e.g., a stripline) that corresponds to a respective portion or quadrant of an emitter, and the pairs of phase shifters can correspond to oppositely-oriented excitable structures on the emitter. In an example, the first and third phase shifters 410A and 410C can correspond to respective striplines of a midfield transmitter.

In an example, the fourth phase shifter 410D can be set to a reference phase shift value, such as zero degrees. In this example, the second phase shifter 410B can be set to 180 degrees, since each pair of signals is offset by 180 degrees. The third phase shifter 410C can be adjusted to maximize power transfer efficiency to an implanted device. The first phase shifter 410A can be configured to maintain a 180 degree offset relative to that imposed by the third phase shifter 410C. Accordingly, when the third phase shifter 410C is set to 90 degrees, the first phase shifter 410A can be set to 270 degrees, and so forth. A phase relationship among or between the four ports or signal paths can therefore be fully defined by the setting of the third phase shifter 410C. Stated differently, the value of the third phase shifter 410C can define a relative phase difference between the pairs of opposing ports in the external source 102.

In one or more examples, a transmit power requirement from the midfield source is Ptt. The signal provided to the power divider 412 has a power of 4*M. The output of the amplifier 408A is about M*P1*Pk. Thus, the power transmitted from the midfield coupler is M* (P1*Pk+P2*Pk+P3*Pk+P4*Pk)=Ptt. Solving for Pk yields Pk=Ptt/(M*(P1+P2+P3+P4)).

The amplitude of a signal at each RF port can be transmitted with the same relative (scaled) amplitude as the signal received at the respective port of the midfield coupler coupled thereto. The gain of the amplifiers 408A-D can be further refined to account for any losses between the transmission and reception of the signal from the midfield coupler. Consider a reception efficiency of η=Pir/Ptt, where Pir is the power received at the implanted receiver. An efficiency (e.g., a maximum efficiency), given a specified phase and amplitude tuning, can be estimated from an amplitude received at the external midfield source from the implantable source. This estimation can be given as η˜(P1+P2+P3+P4)/Pit, where Pit is an original power of a signal from the implanted source. Information about a magnitude of the power transmitted from the implantable device 110 can be communicated as a data signal to the external source 102. In one or more examples, an amplitude of a signal received at an amplifier 408A-D can be scaled according to the determined efficiency, such as to ensure that the implantable device receives sufficient power to perform one or more programmed operation(s) or to delivery a specified therapy using the received power. Given the estimated link efficiency, η, and an implant power (e.g., amplitude) requirement of Pir′, Pk can be scaled as Pk=Pir′/[η(P1+P2+P3+P4)], such as to help ensure that the implant receives adequate power to perform the programmed functions.

Control signals for the phase shifters 410A-D and the amplifiers 408A-D, such as the phase input and gain input, respectively, can be provided by processing circuitry that is not shown in FIG. 9. The circuitry is omitted to not overly complicate or obscure the view provided in FIG. 9. The same or different processing circuitry can be used to update a status of one or more of the switches 404A-D between receive and transmit configurations.

FIG. 10 illustrates generally a diagram of an embodiment of circuitry 500 that comprises the external stimulation controller 152 or the implantable device 110, or other target device.

In an example that comprises the external stimulation controller 152, the circuitry 500 includes a power source 1004. The power source 1004 can include, for example, a single-use or rechargeable battery, or other portable or fixed power source. In an example that comprises the implantable device 110, the circuitry 500 can be configured to receive power wirelessly, for example, via the antenna 108. One or more pad(s) 536 can be electrically connected to the antenna 108. The circuitry 500 can include a tunable matching network 538 to set an impedance of the antenna 108 based on an input impedance of the circuitry 500. The impedance of the antenna 108 can change, for example, due to environmental changes. The tunable matching network 538 can adjust the input impedance of the circuitry 500 based on the varying impedance of the antenna 108. In one or more examples, the impedance of the tunable matching network 538 can be matched to the impedance of the antenna 108. In one or more examples, the impedance of the tunable matching network 538 can be set to cause a portion of a signal incident on the antenna 108 reflect back from the antenna 108, thus creating a backscatter signal.

A transmit-receive (T/R) switch 541 can be used to switch the circuitry 500 from a receive mode (e.g., in which power and/or data signals can be received) to a transmit mode (e.g., in which signals can be transmitted to another device, implanted or external). An active transmitter can operate at an Industrial, Scientific, and Medical (ISM) band of 2.45 GHz or 915 MHz, or the 402 MHz Medical Implant Communication Service (MICS) band for transferring data from the implant. Alternatively, data can be transmitted using a Surface Acoustic Wave (SAW) device that backscatters incident radio frequency (RF) energy to the external device.

The circuitry 500 can include a power meter 542 for detecting an amount of received power at the implanted device, or an amount of power available from the power source 1004. The power meter 542 can provide a power level-indicating signal, and the signal can be used by a digital controller 548 to determine whether power is adequate (e.g., above a specified threshold) for the circuitry to perform some specified function. In an example, a relative value of a signal produced by the power meter 542 can be used to indicate to a user or machine whether an external device (e.g., the source 102) used to power the circuitry 500 is in a suitable location for transferring power and/or data to the target device.

In one or more examples, the circuitry 500 can include a demodulator 544 for demodulating received data signals. Demodulation can include extracting an original information-bearing signal from a modulated carrier signal. In one or more examples, the circuitry 500 can include a rectifier 546 for rectifying a received AC power signal.

Circuitry (e.g., state logic, Boolean logic, or the like) can be integrated into the digital controller 548. The digital controller 548 can be configured to control various functions of the receiver device, such as based on the input(s) from one or more of the power meter 542, demodulator 544, and/or the clock 550. In one or more examples, the digital controller 548 can control which electrode(s) (e.g., E0-E3) are configured as a current sink (anode) and which electrode(s) are configured as a current source (cathode). In one or more examples, the digital controller 548 can control a magnitude of a stimulation pulse produced through the electrode(s).

A charge pump 552 can be used to increase the rectified voltage to a higher voltage level, such as can be suitable for stimulation of the nervous system. The charge pump 552 can use one or more discrete components to store charge for increasing the rectified voltage. In one or more examples, the discrete components include one or more capacitors, such as can be coupled to pad(s) 554. In one or more examples, these capacitors can be used for charge balancing during stimulation, such as to help avoid tissue damage.

Stimulation driver circuitry 556 can provide programmable stimulation through various outputs 534, such as to an electrode array. The stimulation driver circuitry 556 can include impedance measurement circuitry, such as can be used to test for correct positioning of the electrode(s) of the array. The stimulation driver circuitry 556 can be programmed by the digital controller to make an electrode a current source, a current sink, or a shorted signal path. The stimulation driver circuitry 556 can be a voltage or a current driver. The stimulation driver circuitry 556 can include or use a therapy delivery circuitry that is configured to provide electrostimulation signal pulses to one or more electrodes, such as using at least a portion of a received midfield power signal from the external source 102. In one or more examples, the stimulation driver circuitry 556 can provide pulses at frequencies up to about 100 kHz. Pulses at frequencies around 100 kHz can be useful for nerve blocking.

The circuitry 500 can include a memory circuitry 558, such as can include a non-volatile memory circuitry. The memory circuitry 558 can include storage of a device identification, neural recordings, and/or programming parameters, among other implant related data.

The circuitry 500 can include an amplifier 555 and analog digital converter (ADC) 557 to receive signals from the electrode(s). The electrode(s) can sense electricity from nerve signals within the body. The nerve signals can be amplified by the amplifier 555. These amplified signals can be converted to digital signals by the ADC 557. These digital signals can be communicated to an external device. The amplifier 555, in one or more examples, can be a trans-impedance amplifier.

The digital controller 548 can provide data to a modulator/power amplifier 562. The modulator/power amplifier 562 modulates the data onto a carrier wave. The power amplifier 562 increases the magnitude of the modulated waveform to be transmitted.

The modulator/power amplifier 562 can be driven by an oscillator/phase locked loop (PLL) 560. The PLL disciplines the oscillator so that it remains more precise. The oscillator can optionally use a different clock from the clock 550. The oscillator can be configured to generate an RF signal used to transmit data to an external device. A typical frequency range for the oscillator is about 10 kHz to about 2600 MHz (e.g., from 10 kHz to 1000 MHz, from 500 kHz to 1500 kHz, from 10 kHz to 100 kHz, from 50 kHz to 200 kHz, from 100 kHz to 500 kHz, from 100 kHz to 1000 kHz, from 500 kHz to 2 MHz, from 1 MHz to 2 MHz, from 1 MHz to 10 MHz, from 100 MHz to 1000 MHz, from 500 MHz to 800 MHz, overlapping ranges thereof, or any value within the recited ranges). Other frequencies can be used, such as can be dependent on the application. The clock 550 is used for timing of the digital controller 548. A typical frequency of the clock 550 is between about one kilohertz and about one megahertz (e.g., between 1 kHz and 100 kHz, between 10 kHz and 150 kHz, between 100 kHz and 500 kHz, between 400 kHz and 800 kHz, between 500 kHz and 1 MHz, between 750 kHz and 1 MHz, overlapping ranges thereof, or any value within the recited ranges). Other frequencies can be used depending on the application. A faster clock generally uses more power than a slower clock.

In an example, some circuitry of the implantable device can be identically or similarly configured (i.e., uses the same components) as the circuitry in the trial system's external stimulator, e.g., the external stimulation controller 152. By incorporating functionally equivalent components, the trial system offers an authentic simulation of the neurostimulation delivered by the permanent implant (i.e., the “implantable device” discussed herein). This design philosophy ensures that patients and clinicians can accurately evaluate the stimulation's therapeutic potential during the trial phase, and that the trial experience can be substantially replicated using the permanent implant.

A return path for a signal sensed from a nerve is optional. Such a path can include the amplifier 555, the ADC 557, the oscillator/PLL 560, and the modulator/power amplifier 562. Each of these items and connections thereto can optionally be removed.

In one or more examples, the digital controller 548, the amplifier 555, and/or the stimulation driver circuitry 556, among other components of the circuitry 500, can comprise portions of a state machine device. The state machine device can be configured to wirelessly receive power and data signals via the pad(s) 536 and, in response, release or provide an electrostimulation signal via one or more of the outputs 534. In one or more examples, such a state machine device needs not retain information about available electrostimulation settings or vectors, and instead the state machine device can carry out or provide electrostimulation events after, and/or in response to, receipt of instructions from the source 102.

For example, the state machine device can be configured to receive an instruction to deliver a neural electrostimulation therapy signal, such as at a specified time or having some specified signal characteristic (e.g., amplitude, duration, etc.), and the state machine device can respond by initiating or delivering the therapy signal at the specified time and/or with the specified signal characteristic(s). At a subsequent time, the device can receive a subsequent instruction to terminate the therapy, to change a signal characteristic, or to perform some other task. Thus, the device can optionally be configured to be substantially passive, or can be configured to be responsive to received instructions (e.g., contemporaneously received instructions).

Although various general and specific embodiments are described herein, it will be evident that various modifications and changes can be made to these embodiments without departing from the broader spirit and scope of the present disclosure. Accordingly, the specification and drawings are to be regarded in an illustrative rather than a restrictive sense. The accompanying drawings that form a part of this application show, by way of illustration, and not of limitation, specific embodiments in which the subject matter can be practiced. The embodiments illustrated are described in sufficient detail to enable those skilled in the art to practice the teachings disclosed herein. Other embodiments can be used or derived therefrom, such that structural and logical substitutions and changes can be made without departing from the scope of this disclosure. This Detailed Description, therefore, is not to be taken in a limiting sense, and the scope of various embodiments is defined only by the appended claims, along with the full range of equivalents to which such claims are entitled. Specific embodiments or examples are illustrated and described herein, however, it should be appreciated that any arrangement calculated to achieve the same purpose can be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 kHz” includes “10 kHz.” Terms or phrases preceded by a term such as “substantially” or “generally” include the recited term or phrase. For example, “substantially parallel” includes “parallel” and “generally cylindrical” includes cylindrical.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention(s) and embodiments should be determined with reference to the appended claims, along with the full scope of equivalents to which such claims are entitled.

What is claimed is:

1. A neurostimulation trial system comprising:
an external stimulation controller comprising:
a housing;
a processor circuit within the housing;
a signal generator circuit coupled to the processor circuit and configured to generate neurostimulation signals in response to instructions from the processor circuit; and
a transceiver circuit coupled to the processor circuit; and
an external wireless interface device configured to:
receive first stimulation programming instructions from at least one of a clinician device or a patient device; and
in response to the first stimulation programming instructions, communicate second stimulation programming instructions to the transceiver circuit of the external stimulation controller to initiate or adjust a neurostimulation therapy, wherein the neurostimulation therapy comprises the neurostimulation signals generated by the signal generator circuit.

2. The neurostimulation trial system of claim 1, comprising a ground pad configured to adhere the housing of the external stimulation controller directly to patient skin tissue.

3. The neurostimulation trial system of claim 1, comprising at least one trial lead coupled to a lead interface of the external stimulation controller, and wherein the trial lead is configured for implantation at a neural target.

4. The neurostimulation trial system of claim 1, wherein the external wireless interface device is configured to receive the first stimulation programming instructions via a first data communication protocol, and wherein the external wireless interface device is configured to provide the second stimulation programming instructions to the transceiver circuit of the external stimulation controller using a different second data communication protocol.

5. The neurostimulation trial system of claim 1, wherein the first and second stimulation programming instructions comprise the same instructions.

6. The neurostimulation trial system of claim 1, wherein the first and second stimulation programming instructions comprise different instructions.

7. The neurostimulation trial system of claim 1, wherein the external stimulation controller comprises a battery, and wherein each of the processor circuit, the signal generator circuit, and the transceiver circuit is configured to receive power from the battery.

8. The neurostimulation trial system of claim 1, comprising a magnetic switch, wherein at least one function of the stimulation controller is disabled in response to a state change of the magnetic switch.

9. The neurostimulation trial system of claim 8, wherein the magnetic switch is configured to selectively enable or disable generation of the neurostimulation signals.

10. The neurostimulation trial system of claim 1, wherein the external wireless interface device comprises wireless power transmission circuitry configured to communicate power to an implanted device, wherein the wireless power transmission circuitry is deactivated in a trial therapy mode, and wherein the wireless power transmission circuity is activated in a second therapy mode.

11. A treatment system comprising:
a neurostimulation trial system including a body-external stimulation controller and at least one lead configured to provide a first neurostimulation signal from the stimulation controller to a neural target inside a patient body;
a wireless transmitter-receiver device configured to be positioned outside the patient body at a tissue interface; and
an implantable neurostimulation device configured for implantation inside the patient body near the tissue interface, the neurostimulation device comprising a circuitry housing, a pulse generator circuit, and multiple electrodes, wherein the implantable neurostimulation device is configured to receive power wirelessly from the wireless transmitter-receiver device, and wherein the implantable neurostimulation device is configured to use the wirelessly- received power to provide a second neurostimulation signal from the pulse generator circuit to the neural target inside the patient body.

12. The treatment system of claim 11, wherein the body-external stimulation controller includes a second pulse generator circuit that is configured substantially identically to the pulse generator circuit in the implantable neurostimulation device.

13. The treatment system of claim 11, wherein the wireless transmitter-receiver device is configured to use a first wireless communication link to communicate with the body-external stimulation controller, and the wireless transmitter-receiver device is configured to use a different second wireless communication link to communicate with the implantable neurostimulation device.

14. The treatment system of claim 13, wherein the first wireless communication link comprises a nearfield or farfield communication link, and wherein the second wireless communication link comprises a midfield link.

15. The treatment system of claim 11, comprising a programmer device configured to communicate therapy configuration information to the body-external stimulation controller via the wireless transmitter-receiver device.

16. The treatment system of claim 15, wherein the programmer device is configured to communicate the same therapy configuration information or other therapy configuration information to the implantable neurostimulation device.

17. A neuromodulation trial system comprising:
an external stimulator configured to adhere to a patient's skin and to generate neurostimulation signals, wherein the external stimulator includes circuitry that is identical or functionally equivalent to circuitry used in a corresponding permanent implantable neurostimulator;
wherein the external stimulator includes a wireless communication circuit configured to communicate with at least one external device; and
wherein the external stimulator is configured to provide the generated neurostimulation signals to target neural tissue using an implantable lead.

18. The neuromodulation trial system of claim 17, comprising a wireless interface device configured to receive programming instructions from a patient or clinician device and, in response, provide further programming instructions to the external stimulator using the wireless communication circuit.

19. The neuromodulation trial system of claim 18, wherein the wireless interface device is configured to provide the programming instructions to the external stimulator using a first communication link, and wherein the wireless interface device is configured to communicate with the implantable neurostimulator using a different second communication link.

20. The neuromodulation trial system of claim 17, wherein the external device comprises a user interface configured to receive input from the patient or a clinician to adjust the neurostimulation signals, and wherein the external device is configured to communicate a power signal to the permanent implantable neurostimulator.
